(19) United States
(12) Patent Application Publication
(10) Pub. No.: US 2023/0106767 A1
(43) Pub. Date: Apr. 6, 2023

(54) MOVABLE GERMICIDAL ASSEMBLIES FOR DISINFECTION APPARATUSES

(71) Applicant: Anram Holdings, Mississauga (CA)

(72) Inventors: Prakash Valentino RAMANAND, Burlington (CA); Jose Miguel BARRIOS SIERRA, Toronto (CA); Romil Hitenbhai DALVADI, Brampton (CA)

(73) Assignee: Anram Holdings, Mississauga, ON (CA)

(21) Appl. No.: 17/958,215

(22) Filed: Sep. 30, 2022

Related U.S. Application Data
(60) Provisional application No. 63/251,456, filed on Oct. 1, 2021.

(51) Int. Cl. A61L 2/10

(52) U.S. Cl. CPC A61L 2/10; A61L 2202/20; A61L 2202/16; A61L 2202/122; A61L 2202/14

(57) ABSTRACT

Embodiments of the present disclosure provide an apparatus and a method for disinfecting an article. The method includes providing a disinfection chamber inside a cabinet. The disinfection chamber includes a tray moveable between a first position outside the cabinet for placement and retrieval of an article in the tray and a second position within the disinfection chamber for disinfection of the article. The method also includes manipulating, via a controller, a first germicidal assembly movably mounted to the disinfection chamber. The first germicidal assembly includes an articulated arm and a first UV panel mounted thereto, where the articulated arm is manipulated to extend the first UV panel in a first direction towards the article inside the cabinet for disinfecting the article.

### TECHNICAL FIELD

The subject matter generally relates to ultraviolet devices and particularly relates to movable germicidal assemblies for disinfection apparatuses.

### BACKGROUND

Surfaces of portable articles like mobile phones and bags tend to harbour harmful pathogens such as bacteria and viruses. Different disinfection devices employing ultraviolet (UV) light are available in the market for disinfecting such articles. These disinfection devices generally have a housing and a tray for introducing an article into the housing for disinfection. The housing typically has a UV source that irradiates UV light for disinfecting the article. However, the UV source is usually immovably attached to the housing and often fails to radiate the UV light on different surfaces of the article simultaneously, thereby increasing the total time required for complete or adequate article disinfection.

### SUMMARY

In one traditional approach, a disinfection device has a UV source movably attached to a housing. The UV source typically has limited movement that prevents the UV light from being projected on all sides or surfaces of the article simultaneously. Moreover, the UV source typically emits the UV light from a single side relative the article that often results in shadows of the surrounding surfaces falling on to the article to weaken the disinfecting effect of the UV light. As a result, complete or adequate disinfection of the article is either prevented or delayed, causing (1) a net increase in power or battery consumption during a disinfection cycle due to a requirement of prolonged UV emission; (2) an increase in operational costs; and/or (3) user inconvenience and productivity loss because of the article being inaccessible for prolonged periods of disinfection. Therefore, a robust solution is needed to address the above problems.

One embodiment of the present disclosure includes an apparatus for disinfecting an article. The apparatus includes a disinfection chamber inside a cabinet, a tray, and a first germicidal assembly. The tray may be movably attached to the disinfection chamber. The tray may be moveable between a first position outside the cabinet for placement and retrieval of an article in the tray and a second position within the disinfection chamber for disinfection of the article. The first germicidal assembly may also be movably mounted to the disinfection chamber. The first germicidal assembly may include an articulated arm and a first UV panel mounted thereto, where the articulated arm may be configured to extend the first UV panel in a first direction towards the article inside the cabinet for disinfecting the article.

Another embodiment of the present disclosure includes a method of disinfecting an article. The method includes providing a disinfection chamber inside a cabinet. The disinfection chamber may include a tray moveable between a first position outside the cabinet for placement and retrieval of an article in the tray and a second position within the disinfection chamber for disinfection of the article. The method may also include manipulating, via a controller, a first germicidal assembly movably mounted to the disinfection chamber. The first germicidal assembly may include an articulated arm and a first UV panel mounted thereto, where the articulated arm may be manipulated to extend the first UV panel in a first direction towards the article inside the cabinet for disinfecting the article.

The above summary of exemplary embodiments is not intended to describe each disclosed embodiment or every implementation of the present application. Other and further aspects and features of the disclosure would be evident from reading the following detailed description of the embodiments, which are intended to illustrate, not limit, the present application.

### BRIEF DESCRIPTION OF THE DRAWINGS

The illustrated embodiments of the subject matter will be best understood by reference to the drawings, wherein like parts are designated by like numerals throughout. The following description is intended only by way of example, and simply illustrates certain selected embodiments of apparatuses, devices, systems, and processes that are consistent with the subject matter as claimed herein.

FIG. 1 is a front-right perspective view of a mobile disinfection apparatus including a cabinet and a controller, according to an embodiment of the present application.

FIG. 2 is an exemplary disinfection chamber including a tray in a non-extended position for the disinfection apparatus of FIG. 1, according to an embodiment of the present application.

FIG. 3 is an exploded view of the disinfection chamber of FIG. 3 including an exemplary movable top germicidal assembly and an exemplary movable bottom germicidal assembly, according to an embodiment of the present application.

FIG. 4 is a perspective view of an exemplary lift assembly in an extended position for the top germicidal assembly of FIG. 3, according to an embodiment of the present application.

FIG. 5 is a perspective view of the lift assembly of FIG. 4 in a retracted position, according to an embodiment of the present application.

FIGS. 6-7 are inverted perspective views of the top germicidal assembly of FIG. 3 in a retracted state, according to embodiments of the present application.

FIG. 8 is a perspective view of optical sensors for the top germicidal assembly of FIG. 3 in an extended position, according to an embodiment of the present application.

FIG. 9 is an inverted perspective view of the top germicidal assembly of FIG. 3 in an extended position, according to an embodiment of the present application.

FIGS. 10-11 are front-right perspective views of the bottom germicidal assembly of FIG. 3, according to embodiments of the present application.

FIG. 12 is a front-left perspective view of the disinfection apparatus of FIG. 1 without the cabinet and including the disinfection chamber of FIG. 2, according to an embodiment of the present application.

FIG. 13 is a front-left perspective view of the disinfection apparatus of FIG. 12 including a closed door and a retracted tray, according to an embodiment of the present application.

FIGS. 14-15 are front-left perspective views of the disinfection apparatus of FIG. 12 including a partially opened door and a partially extended tray, according to an embodiment of the present application.

FIGS. 16-17 are a front-left perspective views of the first disinfection apparatus of FIG. 12 including the completely opened door and a partially extended tray, according to an embodiment of the present application.

FIGS. 18-19 are front-left perspective views of the disinfection apparatus of FIG. 12 including the completely opened door and a fully-extended tray, according to an embodiment of the present application.

FIG. 20 is a left perspective view of the disinfection chamber of FIG. 2 including the top germicidal assembly of FIG. 3 in a retracted position and the tray in an extended position and without a lateral wall, according to an embodiment of the present application.

FIG. 21 is a left perspective view of the disinfection container of FIG. 20 including the tray in a retracted position under an exemplary movable top UV panel in a retracted state, according to an embodiment of the present application.

FIG. 22 is a perspective view of optical sensors of FIG. 8 over an article requiring disinfection, according to an embodiment of the present application.

FIG. 23 is a perspective view of optical sensors of FIG. 22 detecting the article intercepting the optical field between the optical sensors, according to an embodiment of the present application.

FIG. 24 is a left perspective view of the disinfection chamber of FIG. 21 including the top UV panel of FIG. 21 in an extended state and an exemplary bottom UV panel moving in a direction orthogonal thereto, according to an embodiment of the present application.

FIG. 25 is a front-left perspective view of the disinfection chamber of FIG. 24 including the top UV panel of FIG. 21 moving in a direction orthogonal to the direction of motion of the bottom UV panel of FIG. 24, according to an embodiment of the present application.

### DETAILED DESCRIPTION

The following detailed description is provided with reference to the drawings herein. Exemplary embodiments are provided as illustrative examples to enable those skilled in the art to practice the invention. It will be appreciated that further variations of the concepts and embodiments disclosed herein can be contemplated. The examples of the invention described herein may be used together in different combinations. In the following description, details are set forth in order to provide an understanding of the invention. It will be readily apparent, however, that the invention may be practiced without limitation to all these details. Also, throughout the present application, the terms “a” and “an” are intended to denote at least one of a particular element. The terms “a” and “an” may also denote more than one of a particular element. As used herein, the term “includes” means includes but not limited to, the term “including” means including but not limited to. The term “based on” means based at least in part on, the term “based upon” means based at least in part upon, and the term “such as” means such as but not limited to. The term “approximately” means a variation of +/−5% in a stated number or a value of a stated parameter. Further, in the present application, an embodiment showing a singular component should not be considered limiting; rather, the present application is intended to encompass other embodiments including a plurality of the same or similar component, and vice-versa, unless explicitly stated otherwise herein. The present application also encompasses present and future known equivalents of the components referred to herein.

Embodiments are disclosed in the context of a disinfection apparatus for disinfecting portable articles; however, one having ordinary skill in the art would understand that the concepts and embodiments described herein may be implemented in any other types of disinfection apparatuses including, but not limited to, a large-area or room disinfection apparatus, a handheld disinfection apparatus, and a robotic arm including a germicidal source. Further, the disinfection apparatus, or the concepts and embodiments described herein, may be implemented in any suitable functional apparatus such as a robot, a robotic arm, and a handheld apparatus, or any combinations thereof. The robot may include a machine, or vice versa. The machine may include a vehicle, or vice versa. The functional apparatus may be mobile or made stationary.

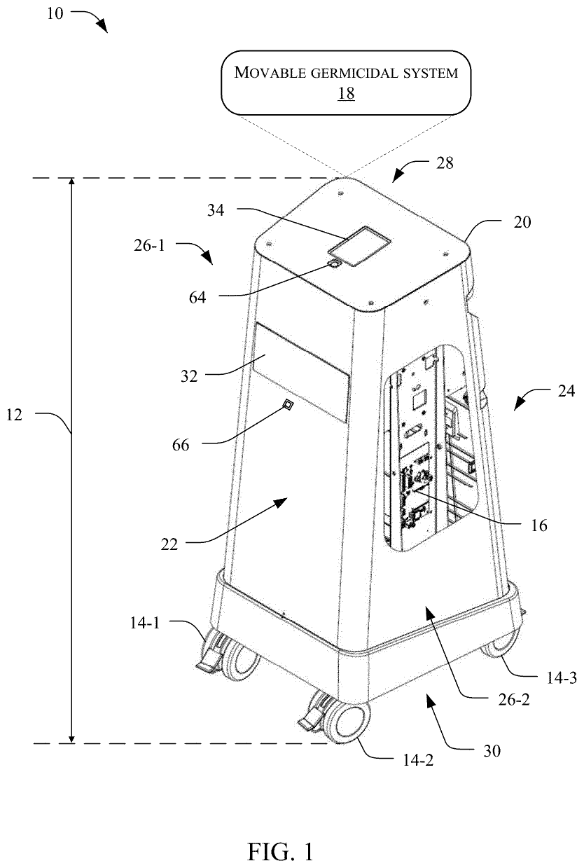

In the illustrated embodiment (FIG. 1), a disinfection apparatus 10 may include a mobile body 12 configured for disinfecting portable articles; however, other embodiments may include the disinfection apparatus 10 having a non-mobile body. The mobile body 12 may include an assembly of parts operable to support, drive, and/or navigate the disinfection apparatus 10 or any components thereof. The mobile body 12 may include a set of wheels such as wheels 14-1, 14-2, and 14-3, hereinafter collectively referred to as wheels 14. The wheels 14 may be motorized or non-motorized. In some examples, the mobile body 12 may additionally, or alternatively, include an autonomous mobile base (not shown). The wheels 14, or the autonomous mobile base, may be configurable for a manual or an automated navigation of the disinfection apparatus 10. The disinfection apparatus 10, and any components thereof including the wheels 14 (or the autonomous mobile base), may be controlled via a control system including a power supply (not shown) located locally thereon. The power supply may include a battery located on the disinfection apparatus 10. Some examples may include the power supply including an internal combustion engine. Other examples may include the power supply, or parts thereof, located external to the disinfection apparatus 10. For instance, the power supply may drive the disinfection apparatus 10 using energy delivered from an external electrical outlet via a power cord.

The control system may further include a controller, such as a controller 16, configured to control predefined or dynamically defined functions and movements of various components of the disinfection apparatus 10. In some examples, the controller 16 may be additionally configured to control devices external or remote to the disinfection apparatus 10. The controller 16 may include an electronic device, or in some examples, an electromechanical device. The controller 16 may include a single device (e.g., a computing device, a processor, or an electronic storage device) or a set of the same or different types of devices. The controller 16 may be implemented in hardware or a suitable combination of hardware and software. The “hardware” may include a combination of discrete electronic or electromechanical components, an integrated circuit, an application-specific integrated circuit, a field programmable gate array, a digital signal processor, or any other suitable hardware known in the art, related art, or developed later. The “software” may include one or more objects, agents, threads, lines of code, subroutines, separate software applications, two or more lines of code or any other suitable software structures operating in one or more software applications.

The controller 16 may be configured to execute machine readable program instructions for processing signals received from various components of the disinfection apparatus 10 or from a remote device in communication therewith. The remote device, in some examples, may include a germicidal source or an apparatus carrying the germicidal source. Other examples may include the remote device including a computing device, a mobile device, a handheld device, or any combinations thereof. Further, the controller 16 may include, for example, microprocessors, microcomputers, microcontrollers, digital signal processors, central processing units, state machines, logic circuits, and/or any devices that may process, manipulate, and output signals based on operational instructions. Among other capabilities, the controller 16 may be configured to fetch and execute computer readable instructions in communication with a storage device (not shown) located locally on the disinfection apparatus 10 or remotely therefrom. The storage device may include any suitable computer-readable medium known in the art, related art, or developed later including, but not limited to, volatile memory (e.g., RAM), non-volatile memory (e.g., flash drive), etc., or any combinations thereof. In some examples, the storage device may be located on the remote device.

In one embodiment, the disinfection apparatus 10 may include a movable germicidal system 18 operating in communication with the controller 16. The movable germicidal system 18 may include a set of one or more distinct germicidal assemblies configured to move or operate independently or in tandem with each other. Other examples may include one germicidal assembly configured to move relative to another germicidal assembly in the movable germicidal system 18. Further, the movable germicidal system 18 may be configured to emit a germicide for disinfecting portable articles, discussed below in greater detail. The germicide may include UV light alone or in combination with any other suitable types of energies or complementing agents for disinfecting the article. Examples of such energies may include, but are not limited to, radio, microwave, x-ray, infrared, visible, or any other specific wavelength or a group of wavelengths in the electromagnetic spectrum. On the other hand, examples of such complementing agents may include, but are not limited to, chemical agents (e.g., alcohols, aldehydes, oxidizing agents, naturally occurring or modified compounds such as titanium dioxide and 2-nitrobenzeldehyde, etc.), physical agents (e.g., heat, pressure, vibration, sound, radiation, plasma, electrical voltage or current, etc.), and biological agents (e.g., tissues or cells of living organisms, plants or plant-based products, organic residues, micro-organisms, etc.) for catalyzing or effecting surface disinfection. The germicide may be selected based on a variety of factors including, but not limited to, an article surface type (e.g., metal, polymer, glass, fluid, organic or inorganic fiber, etc.), an article property (e.g., porosity, permeability, absorptivity, reflectivity, flammability, thermal sensitivity, weight, transparency, transmissivity, etc.), and a pathogen type (e.g., MRSA, Salmonella, E. coli, Clostridium difficile, severe acute respiratory syndrome (SARS) virus or SARS coronavirus, etc.) on the article/surface requiring disinfection. The article may include any electronic or non-electronic item requiring disinfection. Examples of the electronic item may include, but are not limited to, mobile phones, tablets, headsets, magnetic stripe cards or smart cards, pen drives, watches, electronic reading devices, navigation devices, and so on. Examples of the non-electronic articles may include, but are not limited to, pens, facial masks, gloves, jewelry, storage bins, containers, eatables, and so on.

The disinfection apparatus 10 (or the mobile body 12) may further include a cabinet 20 providing a housing to cover one or more operational components (e.g., the movable germicidal system 18, the control system, etc.) of the disinfection apparatus 10. The cabinet 20 may be made up of one or more parts (e.g., cabinet panels) configured to prevent the germicide from passing therethrough. In one example, the cabinet 20 or parts thereof may be made or applied with opaque materials. Other examples may include the cabinet 20, or parts thereof, having filters (e.g., optical filters, air filters, dust filters, ozone filters, etc.) to prevent the germicide from passing therethrough. The cabinet 20 may be made up of any durable, light-weight, fire-retardant, and/or fire-resistant materials known in the art, related art, or developed later including, but not limited to, metals, polymers, alloys, or any combinations thereof. The cabinet 20 may be made rigid; however, in some examples, the cabinet 20 may include parts that are semi-rigid or flexible. The cabinet 20 (or the disinfection apparatus 10) may include a front side 22, a rear side 24, a first lateral side 26-1 and a second lateral side 26-2 (collectively referred to as lateral sides 26), a top side 28, and a bottom side 30. The front side 22 may include a front opening (not shown) covered by a door 32. In one example, the front opening may lead into a distinct disinfection chamber 40 located within the cabinet 20. The disinfection chamber 40 may be configured for receiving articles requiring disinfection.

The top side 28 may include a display unit 34 on an outer surface of the cabinet 20 (or the disinfection apparatus 10). The display unit 34 may provide information pertaining to an operation of the disinfection apparatus 10. In one example, the display unit 34 may represent or include an interactive display screen allowing an operator to access, control, or dynamically define different functions of the disinfection apparatus 10. The display unit 34 may display a dashboard providing a list of functions, modes, parameters, avatars, etc., which the operator may select or modify for a desired operation of the disinfection apparatus 10. Other examples may include the display unit 34 including or operating in communication with a variety of tangible indicators (e.g., light sources, vibrators, speakers, etc.) or virtual indicators displayable on the dashboard, or any other components operationally connected to the display unit 34 and/or the controller 16. These tangible indicators, or the virtual indicators, may indicate different operational aspects of the disinfection apparatus 10. Examples of such virtual indicators may include, but are not limited to, numeric indicators, alphanumeric indicators, and non-alphanumeric indicators such as different colors, different color luminance, different patterns, different textures, and different graphical objects. Examples of these operational aspects may include, but are not limited to, (i) values of operational parameters such as frequency, wavelength, duration, energy, and dose, (ii) different modes of operation and/or a selected mode in operation, (iii) operational states of different components, (iv) statuses of various operational tasks such as a disinfection cycle, network data transfer, and remote administration, (v) a number of disinfection cycles completed or run on the disinfection apparatus 10, and (vi) a number of flashes or pulses of the germicide (e.g., UV light) projected by the corresponding germicidal source or the disinfection apparatus 10. Some examples may include the display unit 34 located remote from the top side 28 and positioned on any other side or component of the disinfection apparatus 10. Other examples may include the display unit 34 located on the remote device in communication therewith and/or the controller 16.

The cabinet 20 may also include one or more vents (not shown) to assist in cooling operational components (e.g., the movable germicidal system 18, the control system, etc.) of the disinfection apparatus 10. The vents may be located on any of the lateral sides 26, the front side 22, the rear side 24, and the bottom side 30. The vents may assist in dissipating the heat or warm air around the operational components into the ambient environment. In some examples, the vents may also allow an intake of fresh air (or cool air) into the cabinet 20, or any other housings or portions of the disinfection apparatus 10, for cooling operational components located therein. The vents may be configured in a manner (e.g., downwardly slanted and/or covered with a suitable filter) that prevents the germicide to escape from the corresponding housing, such as the cabinet 20, and the disinfection apparatus 10.

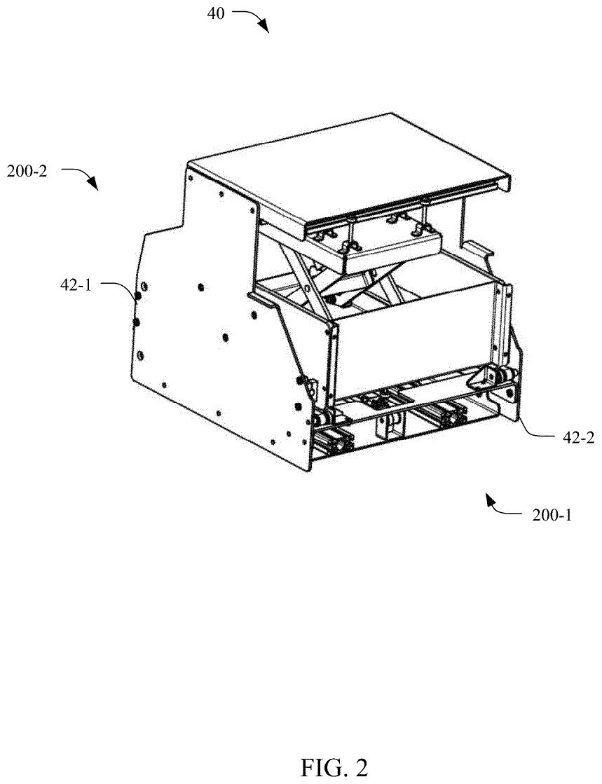

In one embodiment, as illustrated in FIG. 2, the disinfection apparatus 10 may include the disinfection chamber 40 distinct or separate from the cabinet 20. The disinfection chamber 40 may refer to a component (or a bounded region) of the disinfection apparatus 10, where such component or region may be configured to receive articles requiring disinfection and substantially irradiate the germicide on to the received articles. In one example, the disinfection chamber 40 may be configured as a single component (or a set of different components assembled together) located within the cabinet 20. However, some examples may include the cabinet 20, alone or in combination with another component of the disinfection apparatus 10, defining the disinfection chamber 40. Other examples may include a distinct housing carrying the disinfection chamber 40 located internal or external to the cabinet 20 in the mobile body 12 (or the disinfection apparatus 10).

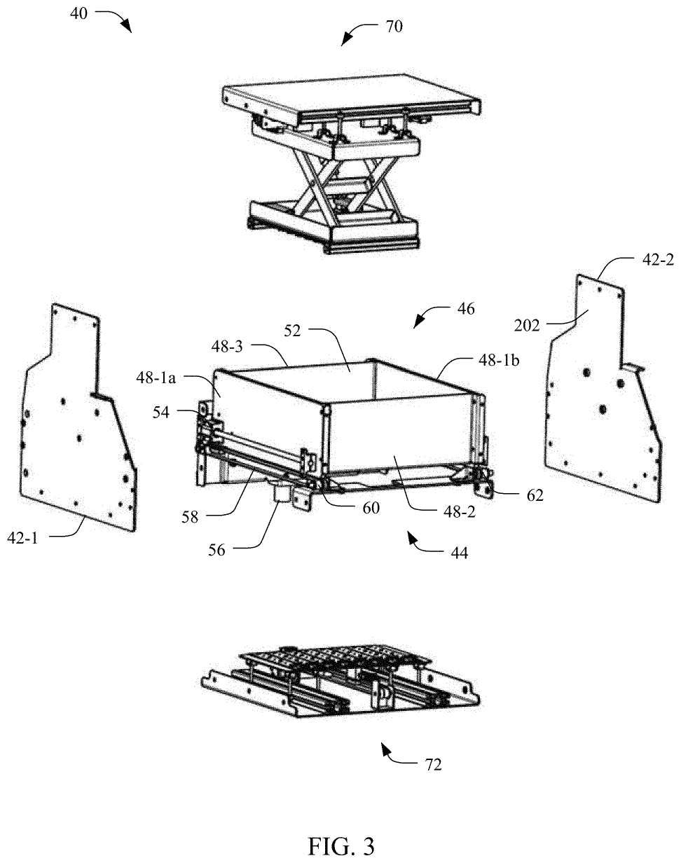

The disinfection chamber 40 may be removably mounted to the disinfection apparatus 10, discussed below in greater detail. As illustrated in in FIG. 3, in one example, the disinfection chamber 40 may include a first side plate 42-1 and a second side plate 42-2 (hereinafter collectively referred to as side plates 42), a container assembly 44, and the movable germicidal system 18. The side plates 42 may be configured to provide a support structure for removably mounting the container assembly 44 and the movable germicidal system 18. The side plates 42 may extend between a front side 200-1 and a rear side 200-2 of the disinfection chamber 40. The side plates 42 may be made of any suitable materials known in the art (e.g., metals, alloys, polymers, etc.) configured to have reflective properties. Each of the side plates 42 may have an inner plate surface, such as an inner plate surface 202, oriented towards the movable germicidal system 18 and/or the container assembly 44. The inner plate surface 202 being treated or overlaid with any suitable materials known in the art that enable or enhance germicide reflectivity (e.g., reflective paint, hydrophobic materials, mirrors, lens, etc.). Other examples may include the inner plate surface, such as the inner plate surface 202, being polished or textured for reflecting or redirecting the germicide towards the container assembly 44 mounted to the side plates 42.

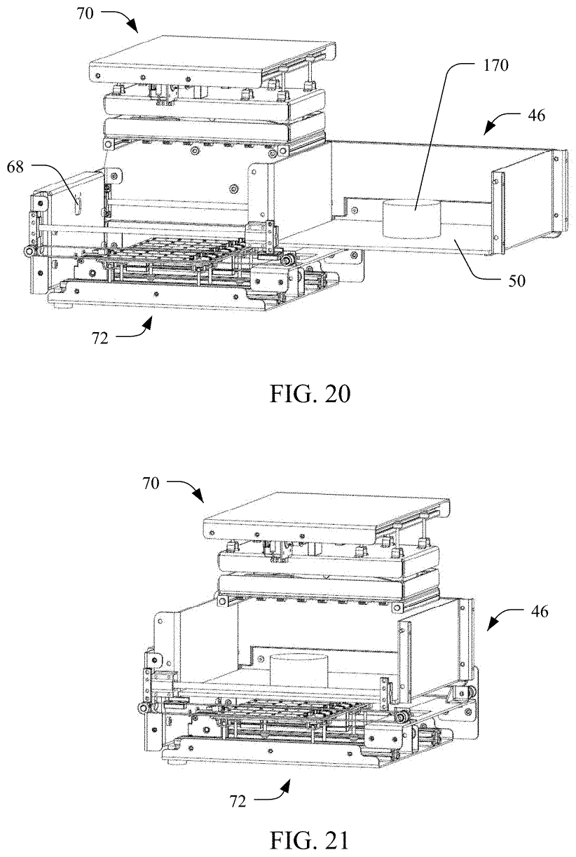

The container assembly 44 may include a tray 46 providing a platform for supporting or carrying the article requiring disinfection. The tray 46 may include a first lateral wall 48-1a and a second lateral wall 48-1b (hereinafter collectively referred to as lateral walls 48-1), a front wall 48-2, a rear wall 48-3 (hereinafter collectively referred to as tray walls 48) and a tray base 50 (shown later in FIG. 20). Each of the tray walls 48 may be mounted or formed integral to the tray base 50 for receiving the articles for disinfection. The tray walls 48 may extend perpendicular to the tray base 50 (or a horizontal axis of the disinfection apparatus 10); however, in some examples, one or more of the tray walls 48 may be arranged at a predefined wall angle to facilitate in redirecting the germicide towards the tray base 50 supporting or carrying the articles requiring disinfection. The wall angle may range from approximately 90 degrees to approximately 45 degrees. In some examples, any of the tray walls 48 may be slanted inwards towards the tray base 50 or outwards therefrom. Each of the tray walls 48 may have the same height; however, some examples may include at least one of the tray walls 48 being shorter or longer (or taller) than the remaining tray walls 48 to assist in directing or redirecting the germicide towards the tray base 50. The tray walls 48 may have a height ranging from approximately 1 inch (i.e., 2.5 cm) to approximately 6 inches (i.e., 15 cm); however, the height greater than approximately 6 inches (i.e., 15 cm) may also be contemplated depending on a space available within the disinfection chamber 40.

The tray base 50 may be surrounded by the tray walls 48. Each of the tray walls 48 may be made up of any suitable materials known in the art (e.g., metals, alloys, polymers, etc.) configured to have reflective properties. The tray walls 48 may have inner wall surfaces, such as an inner tray wall 52, oriented towards the tray base 50. The inner tray wall 52 may be treated or overlaid with suitable reflective materials, such as those mentioned above, for redirecting or reflecting the germicide substantially towards the tray base 50. Some examples may include the inner tray wall 52 being additionally polished or textured. Further, the tray base 50 may be made up of any suitable material (e.g., quartz) that may be permeable to the germicide. In some examples, the tray base 50 may also include gaps or perforations (not shown) allowing penetration or transmission of the germicide therethrough. In some examples, the tray base 50 may include partitions (not shown) for receiving the articles. The partitions may be formed by one or more inner walls (not shown) having reflective surfaces. The inner walls may extend at a predefined wall angle, such as that mentioned above, from the tray base 50. The inner walls may be surrounded by the tray walls 48. The inner walls may have a height less (or greater, in some examples) than that of the tray walls 48.

The container assembly 44 may also include a sliding mechanism for moving the tray 46 between a non-extended position inside the disinfection chamber 40 and an extended position outside the disinfection chamber 40 (or the cabinet 20). In one embodiment, the sliding mechanism may be configured to include a leadscrew mechanism and a belt-clamp arrangement for moving the tray 46 between the non-extended position and the extended position (hereinafter collectively referred to as operative positions). The leadscrew mechanism may include at least one leadscrew for moving the tray 46 between the operative positions. For example, as illustrated, the tray 46 may include a leadscrew 54 connected to the first lateral wall 48-1a; however, in some examples, another leadscrew may also be connected to the second lateral wall 48-1b. The leadscrew 54 may be operationally connected to a tray motor 56. The leadscrew 54 may be configured to convert a rotary motion of the tray motor 56 to a linear motion of the tray 46. The tray motor 56 may operate in communication with the controller 16 to selectively drive the leadscrew 54 for moving the tray 46 between the operative positions.

On the other hand, the belt-clamp arrangement may include a system of belts and pulleys to assist in sliding the tray 46 between the operative positions. For example, the tray 46 may be movably mounted on a set of left pulleys and a set of right pulleys (collectively referred to as support pulleys). Each of the support pulleys may provide a surface on which the tray 46 may slide. The left pulleys may include a belt such as a tray belt 58 mounted thereon. Similarly, the right pulleys may include another tray belt mounted thereon. Each of these tray belts such as the tray belt 58 may be attached to the tray 46 via tray clamps (not shown). The tray clamps may be removably attached to both the tray belts and an underside of the tray 46. The tray belts may assist in sliding the tray 46 over the support pulleys such as a first front pulley 60 and a second front pulley 62; however, some examples may include the tray belts being driven by the tray motor 56 in tandem with the leadscrew 54 for moving the tray 46 in and out of the disinfection chamber 40 (and the cabinet 20). It will be appreciated by a person having ordinary skill in the art that any other suitable types of sliding mechanisms known in the art, related art, or developed later including a ball-screw mechanism may be implemented for controllably and selectively moving the tray 46 in and out of the disinfection chamber 40 (or the cabinet 20).

Different positions of the tray 46 may be selectively controlled and monitored by the controller 16 using any of a variety of sensors. For example, as illustrated in FIG. 1, the disinfection apparatus 10 may include a top sensor 64 and a front sensor 66. The top sensor 64 (e.g., a fingerprint sensor, a camera, a proximity sensor such as ultrasonic sensor, a laser or light sensor, and a radiofrequency or RFID sensor, etc.) may be located on the top side 28 of the cabinet 20 (or the disinfection apparatus 10); however, any other suitable locations for the top sensor 64 may also be contemplated. The top sensor 64 may assist in controlling or maneuvering a forward/outward motion or a backward/inward motion of the tray 46. In the forward/outward motion, the tray 46 may move outward and away from the disinfection chamber 40 (or the cabinet 20). In the backward/inward motion, the tray 46 may move towards the disinfection chamber 40 (or the cabinet 20).

The front sensor 66 (e.g., a camera, a proximity sensor such as those mentioned above, etc.) may be positioned proximate to the door 32 or the front opening. The front sensor 66 may be located on the front side 22 of the cabinet 20 (or the disinfection apparatus 10). The front sensor 66 may assist in detecting a presence of an object or a user in a forward/outward moving path of the tray 46 outside the cabinet 20 (or the disinfection apparatus 10). Similarly, the disinfection apparatus 10 may also include a rear sensor 68 (shown later in FIG. 20) located rear to the tray 46. For example, the rear sensor 68 (e.g., a camera, a mechanical contact switch, a proximity sensor such as those mentioned above, etc.) may be mounted to the cabinet 20 proximate to the rear wall 48-3 of the tray 46. Other examples may include the rear sensor 68 being mounted to the disinfection chamber 40 proximate to the rear wall 48-3 of the tray 46. The rear sensor 68 may be configured to monitor a position of the tray 46 and assist in manipulating (e.g., start or stop) the tray motor 56 for driving the tray 46 up to an intended distance outside and within the disinfection chamber 40. Each of the front sensor 66, the top sensor 64, the rear sensor 68, the tray motor 56 and any components connected thereto, such as the display unit 34 and the tray 46, may be controlled by the controller 16. The tray 46 may be mounted proximate to the movable germicidal system 18 within the disinfection chamber 40.

In one embodiment, the movable germicidal system 18 may be configured to (i) project a germicide within the disinfection chamber 40 and (ii) reposition at least one germicidal source within the disinfection chamber 40 relative to (1) an article received therein, (2) a surface carrying or supporting the article within the disinfection chamber 40, and (3) another germicidal source within the disinfection chamber 40, or any combinations thereof. The movable germicidal system 18 may include a set of one or more distinct germicidal assemblies. For example, as illustrated in FIG. 3, the movable germicidal system 18 may include a movable top germicidal assembly 70 and a movable bottom germicidal assembly 72 located apart from each other. Each of the top germicidal assembly 70 and the bottom germicidal assembly 72 may be configured to emit the same or different types of germicide such as those mentioned above. The top germicidal assembly 70 may be configured to operate or move independent of the bottom germicidal assembly 72; however, some examples may include the top germicidal assembly 70 being configured to operate or move in tandem with the bottom germicidal assembly 72. The top germicidal assembly 70 and the bottom germicidal assembly 72 may be disposed on opposite sides of the tray 46. For example, the top germicidal assembly 70 may be attached to the side plates 42 and located over the tray 46. The bottom germicidal assembly 72 may be attached to the side plates 42 and located under the tray 46. However, some examples may include the top germicidal assembly 70 and the bottom germicidal assembly 72 located lateral to the tray 46. For instance, the top germicidal assembly 70 may be attached to only the first side plate 42-1 and located parallel to the first lateral wall 48-1a of the tray 46. Similarly, in another instance, the bottom germicidal assembly 72 may be attached to only the second side plate 42-2 and located parallel to the opposite second lateral wall 48-1b of the tray 46. Some other examples may include the top germicidal assembly 70 and the bottom germicidal assembly 72 being disposed on the same side of the tray 46. For instance, both the top germicidal assembly 70 and the bottom germicidal assembly 72 may be located over the tray 46 or under the tray 46. Other examples may include the top germicidal assembly 70 and the bottom germicidal assembly 72 being assembled together or combined into a single unit.

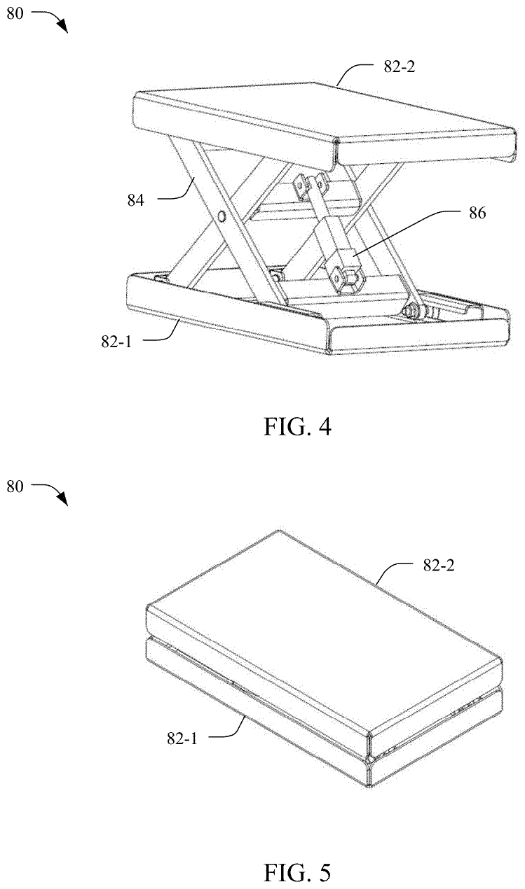

As illustrated in FIG. 4, the top germicidal assembly 70 may include a lift assembly 80, in one embodiment. The lift assembly 80 may include a first lift panel 82-1 and a lift panels 82 (hereinafter collectively referred to as lift panels 82). Each of the lift panels 82 may provide a surface for mounting one or more components thereto. The lift assembly 80 may further include an articulated arm 84 movably connected between the lift panels 82. The articulated arm 84 may be implemented as an articulating scissor arm; however, some examples may include the articulated arm 84 being additionally or alternatively implemented as a telescopic arm. Other examples may include the articulated arm 84 implemented as a pivotable telescopic arm. The articulated arm 84 may have one end connected to the first lift panel 82-1 and an opposing end connected to the lift panels 82. Further, the articulated arm 84 may include a linear actuator 86 configured to extend and retract the articulated arm 84; however, some examples may include any other types of actuators known in the art including a rotary actuator. The linear actuator 86 may be controlled by the controller 16. The linear actuator 86 may drive the articulated arm 84 to selectively move the lift panels 82 between an extended position and a retracted position. In the extended position (FIG. 4), the lift panels 82 may have a gap therebetween greater than a threshold value. In one example, the threshold value may be approximately 0.5 inches (i.e., 1.2 cm) based on the tray walls 48 having the height of approximately 6 inches (i.e., 15 cm); however, other examples may include the threshold value greater than approximately 0.5 inches (i.e., 1.2 cm) up to approximately 3 inches (i.e., 7.5 cm). The linear actuator 86 may drive the articulated arm 84 for moving the lift panels 82 away from each other in the extended position. Further, in the retracted position (FIG. 5), the lift panels 82 may have the gap therebetween less than or equal to the threshold value. The linear actuator 86 may drive the articulated arm 84 for moving the lift panels 82 close to each other in the retracted position. The lift assembly 80 including the lift panels 82 may be mounted to the top germicidal assembly 70; however, some examples may include the lift assembly 80 being alternatively, or additionally, mounted to the bottom germicidal assembly 72.

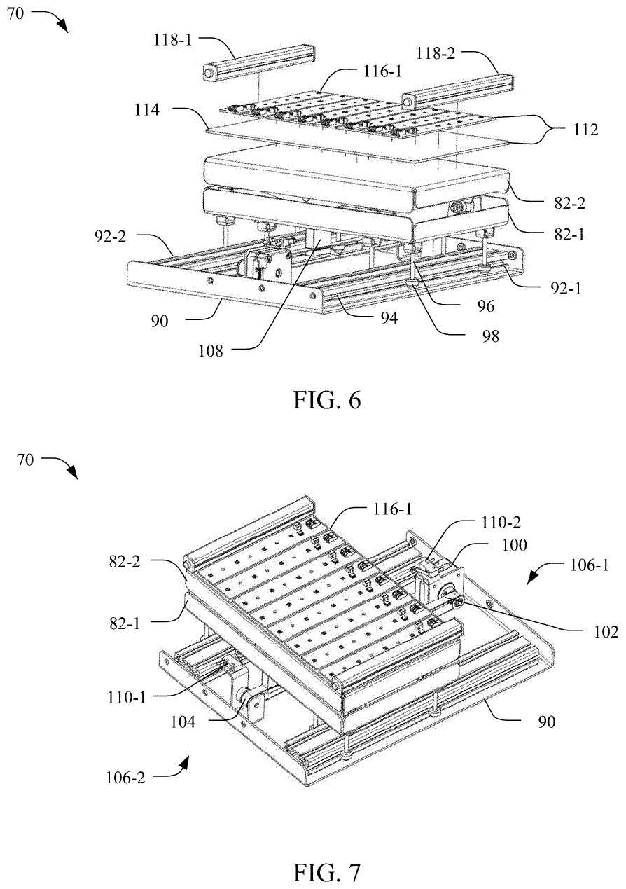

In one embodiment, as illustrated in FIG. 6, the top germicidal assembly 70 may include a top support plate 90 configured and attached to the side plates 42 for support and stability. FIGS. 6-7 illustrate inverted perspective views of the top germicidal assembly 70. The top support plate 90 may include the lift assembly 80 movably mounted thereto. The lift assembly 80 may be mounted across a length of the top support plate 90. The lift assembly 80 may be also positioned within the boundaries of the top support plate 90; however, some examples may include portions (e.g., lift panels 82) of the lift assembly 80 extending outward from a boundary of the top support plate 90. The lift assembly 80 (or the lift panels 82) may have a length substantially the same as a width of the top support plate 90. Further, the top support plate 90 may include a first top rail 92-1 and a second top rail 92-2 (hereinafter collectively referred to as top rails 92). Each of the top rails 92 may include slots such as a first slot 94. The slots may extend along a substantial length (e.g., entire length) of the corresponding top rails 92. The slots may be configured to movably mount one of the lift panels 82 thereto via shouldered screws; however, any other suitable connection mechanisms known in the art may also be contemplated. In one example, the first lift panel 82-1 of the lift assembly 80 may implement multiple shouldered screws such as a first shouldered screw 96. Each of the shouldered screws, such as the first shouldered screw 96, may include one end connected to the first lift panel 82-1 and the other end may include a wheel, such as a first guide wheel 98, configured for being received in one of the slots, such as the first slot 94. The first guide wheel 98 may movably engage with the first slot 94 to assist in moving the first lift panel 82-1, and hence, the lift assembly 80, along a length of the corresponding top rail 92-1 (or the top support plate 90).

The top support plate 90 may further include a top motor 100 controlled by the controller 16. The top motor 100 may be configured to drive the lift assembly 80 along the top rails 92. In one example, the top motor 100 may be connected to a top belt 102 rotating over a top idler pulley 104 (shown in FIG. 7). The top motor 100 may be mounted proximate to a first end 106-1 of the top support plate 90, and the top idler pulley 104 may be mounted proximate to a second end 106-2 of the top support plate 90. In one example, the first end 106-1 of the top support plate 90 may be located proximate to the first lateral side 26-1 of the cabinet 20 (or the disinfection apparatus 10), and the second end 106-2 of the top support plate 90 may be located proximate to the second lateral side 26-2 of the cabinet 20 (or the disinfection apparatus 10). Further, the top belt 102 may extend longitudinally between the first end 106-1 and the second end 106-2 (hereinafter collectively referred to as opposing ends 106) of the top support plate 90. The top motor 100 may be configured to rotate the top belt 102 clockwise and anticlockwise over the top idler pulley 104.

The top belt 102 may be also connected to the first lift panel 82-1 via a top clamp 108 for a tandem motion therebetween. The top clamp 108 may be removably attached to both the first lift panel 82-1 and the top belt 102. The top clamp 108 may assist in moving the first lift panel 82-1 (or the lift assembly 80) based on a movement of the top belt 102 while allowing for no relative movement between the first lift panel 82-1 (or the lift assembly 80) and the top belt 102. The top belt 102 may be moved or rotated by the top motor 100 to selectively move the first lift panel 82-1 (or the lift assembly 80) between the opposing ends 106 of the top support plate 90 along a horizontal axis, e.g., the x-axis. For example, the top motor 100 may rotate the top belt 102 clockwise over the top idler pulley 104 for moving the first lift panel 82-1 (and the lift assembly 80) towards the first end 106-1 of the top support plate 90 (or towards the top motor 100). Similarly, the top motor 100 may rotate the top belt 102 anticlockwise over the top idler pulley 104 for moving the first lift panel 82-1 (and the lift assembly 80) towards the second end 106-2 of the top support plate 90 (or away from the top motor 100). The first lift panel 82-1 (and the lift assembly 80) may be moved parallel to the top rails 92 (or a length of the top support plate 90). Upon being moved, the first lift panel 82-1 (and the lift assembly 80) may slide along the top rails 92 via the guide wheels 14 (such as the first guide wheel 98) engaging with the corresponding slots such as the first slot 94.

Different positions of the lift panels 82 (and the lift assembly 80) between the opposing ends 106 (e.g., along the x-axis) of the top support plate 90 may be determined by the controller 16 via sensors. In one embodiment, the top support plate 90 may include a first top position sensor 110-1 and a second top position sensor 110-2 (hereinafter collectively referred to as the top position sensors 110). Each of the top position sensors 110 may be mounted to a support bracket coupled or attached to the top support plate 90. In one example, the first top position sensor 110-1 may be mounted proximate to the first end 106-1 of the top support plate 90 (or the top motor 100). The second top position sensor 110-2 may be located proximate to the second end 106-2 of the top support plate 90 (or the top idler pulley 104). Each of the top position sensors 110 may be configured as an electromechanical contact switch; however, some examples may include any other suitable types of sensors known in the art, related art, or developed later including physical sensors and wireless sensors such as optical sensors.

The top position sensors 110 may operate in communication with the controller 16 for determining different positions of the lift panels 82 (or the lift assembly 80). For example, based on a contact between the first top position sensor 110-1 and the first lift panel 82-1 (or the lift assembly 80), the first top position sensor 110-1 may be configured to send a first top signal to the controller 16. The first top signal may indicate to the controller 16 that the lift panels 82 (or the lift assembly 80) may be located proximate to the first end 106-1 of the top support plate 90 (or the top motor 100). Similarly, based on a contact between the second top position sensor 110-2 and the first lift panel 82-1 (or the lift assembly 80), the second top position sensor 110-2 may be configured to send a second top signal to the controller 16. The second top signal may indicate to the controller 16 that the lift panels 82 (or the lift assembly 80) may be located proximate to the second end 106-2 of the top support plate 90 (or the top idler pulley 104). When no signal may be received from any of the top position sensors 110, the controller 16 may determine the lift panels 82 (or the lift assembly 80) being located in-between the opposing ends 106 of the top support plate 90. Opposite the first lift panel 82-1, the lift assembly 80 may include a top UV panel 112 configured to project UV light.

As illustrated in FIG. 6, the top UV panel 112 may include a top mounting plate 114 and a top germicidal source 116-1 mounted thereto. In some examples, the top UV panel 112 may also include a fan (not shown) mounted thereto for cooling the top germicidal source 116-1 and other surrounding components. In the illustrated example, the top germicidal source 116-1 includes strips of one or more UV light emitting diodes (LEDs) configured to emit pulses of UV light; however, some examples may include UV lamps or bulbs configured for pulsed or continuous emission of the UV light. The UV LEDs may be configured to emit the UV light of a predetermined energy within a predetermined germicidal wavelength range including 100-280 nm (i.e., UV type C); 280-315 nm (i.e., UV type B); 200-300 nm (i.e., middle UV); and 122-200 nm (i.e., far UV), or any combinations thereof. Other wavelength ranges may also be employed, including those providing ionizing radiation (e.g., extreme UV, with a wavelength of 10-120 nm). In some embodiments, the UV LEDs may be configured by the controller 16 to have a pulse frequency and/or pulse duration that may cause the emitted pulsed UV light to appear as continuous to a human eye. In some other examples, the top germicidal source 116-1 may additionally include other types of radiation sources and/or non-radiation sources providing the complementing agents including those mentioned above for initiating, catalyzing, or effecting disinfection of the intended article. In some examples, the complementing agents may be germicidal in nature. Further, as illustrated in FIG. 7, the top mounting plate 114 may be removably mounted to the lift panels 82 of the lift assembly 80. The top mounting plate 114 may assist in easy wiring, maintenance, and replacement of the top germicidal source 116-1 therefrom.

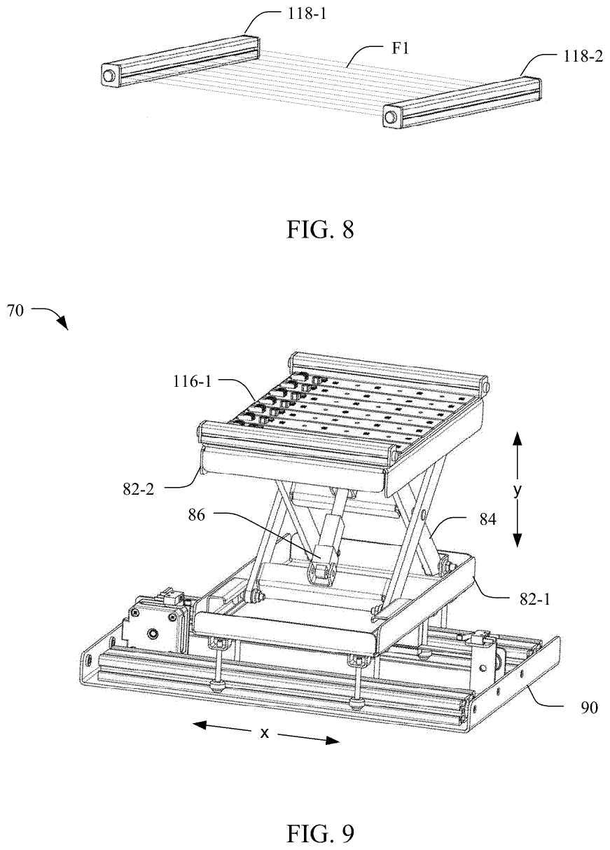

In addition to the top germicidal source 116-1, the top mounting plate 114 may include sensors mounted thereto. For example, the top mounting plate 114 may include an optical transmitter 118-1 and an optical receiver 118-2 (hereinafter collectively referred to as optical sensors 118). The optical sensors 118 may be mounted to the top mounting plate 114 with the top germicidal source 116-1 located therebetween. The top UV panel 112 including the optical sensors 118 and the top germicidal source 116-1 may be oriented towards the tray 46 (or the tray base 50) inside the disinfection chamber 40. Further, the optical sensors 118 may be mounted in the same horizontal plane or within the line of sight of each other. The optical sensors 118 may operate in communication with the controller 16. The optical transmitter 118-1 may be configured to transmit optical signals to the optical receiver 118-2, thereby creating a continuous optical field F1 therebetween, as shown in FIG. 8. In one example, the optical receiver 118-2 may be configured to send a detection signal to the controller 16 based on any interference in the continuous optical field F1; however, other examples may include the optical transmitter 118-1 sending the detection signal to the controller 16 based on such interference. The detection signal (and the interference) may indicate a presence of an article to the controller 16. In one example, the optical transmitter 118-1 may be elongated to have a length, a width, and a thickness (or height) substantially the same as those of the optical receiver 118-2. In some examples, as illustrated, the optical sensors 118 may have a thickness (or height) greater than that of the top germicidal source 116-1. This difference in thicknesses (or heights) of the top germicidal source 116-1 and the optical sensors 118 may preclude any inadvertent interference of the optical field F1, thereby preventing a false object detection by the controller 16.

The top UV panel 112 (or the lift panels 82) may be configured to transition between a retracted state and an extended state (hereinafter collectively referred to as operational states). In the retracted state, as illustrated in FIG. 7, the top UV panel 112 (or the lift panels 82) may be positioned proximate to the top support plate 90 (or the first lift panel 82-1) with the lift panels 82 arranged in the retracted position. In the extended state, as illustrated in FIG. 9, the lift assembly 80 may have the articulated arm 84 extended for moving the top UV panel 112 (and the lift panels 82) in a direction (e.g., along a vertical axis, y-axis) orthogonal (or perpendicular) to the top support plate 90; however, some examples may include the top UV panel 112 being extended in a direction at any angle less than approximately 90 degrees with respect to a horizontal axis of the top support plate 90 (or the disinfection chamber 40). As a result, the top UV panel 112 (or the lift panels 82) may move and position away from the top support plate 90 (or the first lift panel 82-1) when the lift panels 82 may be arranged in the extended position. In some examples, the top UV panel 112 may be moved parallel to the top rails 92 (or a length of the top support plate 90), e.g., along the horizontal axis, x-axis, during any of the operational states based on the underlying lift assembly 80 being moved by the top motor 100, as discussed above. For instance, the lift assembly 80 may be moved parallel to the top rails 92 while moving the top UV panel 112 from the retracted state to the extended state, or vice versa.

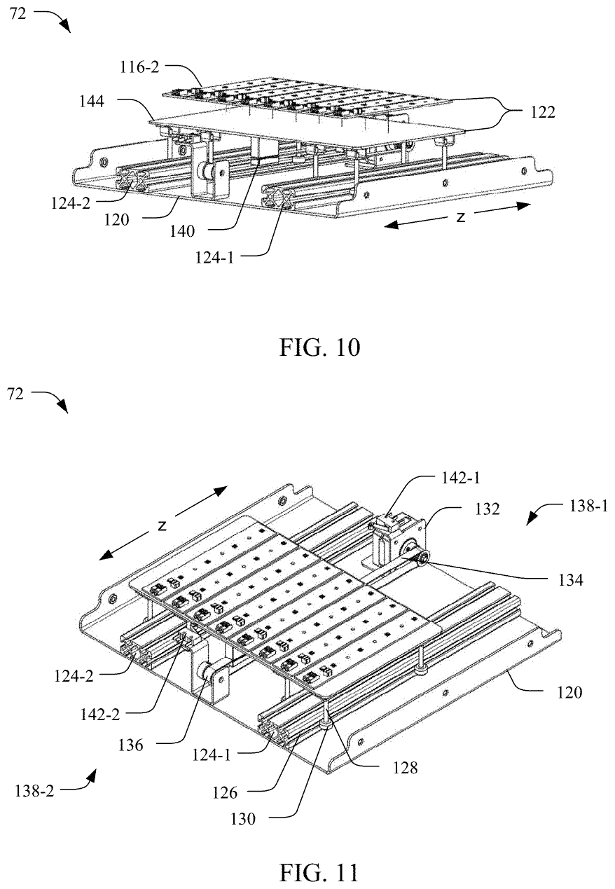

Similar to the top UV panel 112, the bottom germicidal assembly 72 may include a bottom UV panel 122 and a bottom support plate 120. In one embodiment, as illustrated in FIG. 10, the bottom UV panel 122 may be movably mounted to the bottom support plate 120. The bottom support plate 120 may be configured and attached to the side plates 42 for support and stability. The bottom support plate 120 may include a first bottom rail 124-1 and a second bottom rail 124-2 (hereinafter collectively referred to as bottom rails 124). The bottom rails 124 may extend, e.g., along a horizontal axis, z-axis, perpendicular to the top rails 92 inside the disinfection chamber 40. Similar to the top rails 92, each of the bottom rails 124 may include slots such as a second slot 126. The slots may extend along a substantial length (e.g., entire length) of the corresponding bottom rails 124. The slots may be configured to movably mount the bottom UV panel 122 thereto via shouldered screws; however, any other suitable connection mechanisms known in the art may also be contemplated. In one example, the bottom UV panel 122 may implement multiple shouldered screws such as a second shouldered screw 128. Each of these shouldered screws, such as the second shouldered screw 128, may include one end connected to the bottom UV panel 122 and the other end may include a wheel, such as a second guide wheel 130, configured for being received in one of the slots, such as the second slot 126. The second guide wheel 130 may movably engage with the second slot 126 to assist in moving the bottom UV panel 122 along a length of the corresponding bottom rail 124-1 (or the bottom support plate 120) along the z-axis. The bottom UV panel 122 may be mounted across a length of the bottom support plate 120. The bottom UV panel 122 may be positioned within the boundaries of the bottom support plate 120; however, some examples may include portions of the bottom UV panel 122 extending outward from a boundary of the bottom support plate 120. The bottom UV panel 122 may have a length substantially the same as a width of the bottom support plate 120.

As illustrated in FIG. 11, the bottom support plate 120 may further include a bottom motor 132 controlled by the controller 16. The bottom motor 132 may be configured to drive the bottom UV panel 122 along the bottom rails 124. For example, the bottom motor 132 may be connected to a bottom belt 134 rotating over a bottom idler pulley 136. The bottom motor 132 may be mounted proximate to a first end 138-1 of the bottom support plate 120 and the bottom idler pulley 136 may be mounted proximate to a second end 138-2 of the bottom support plate 120. The first end 138-1 of the bottom support plate 120 may be located proximate to the rear side 24 of the cabinet 20 (or the disinfection apparatus 10), and the second end 138-2 of the bottom support plate 120 may be located proximate to the front side 22 of the cabinet 20 (or the disinfection apparatus 10). Further, the bottom belt 134 may extend longitudinally between the first end and the second end (hereinafter collectively referred to as opposing ends) of the bottom support plate 120. The bottom motor 132 may be configured to rotate the bottom belt 134 clockwise and anticlockwise over the bottom idler pulley 136.

The bottom belt 134 may be also connected to the bottom UV panel 122 via a bottom clamp 140 for a tandem motion therebetween. As illustrated in FIG. 10, the bottom clamp 140 may be removably attached to both the bottom UV panel 122 and the bottom belt 134. The bottom clamp 140 may assist in moving the bottom UV panel 122 based on a movement of the bottom belt 134 while allowing for no relative movement between the bottom belt 134 and the bottom UV panel 122. The bottom belt 134 may be rotated by the bottom motor 132 to selectively move the bottom UV panel 122 between the opposing ends of the bottom support plate 120. For example, the bottom motor 132 may rotate the bottom belt 134 clockwise over the bottom idler pulley 136 for moving the bottom UV panel 122 towards the first end 138-1 of the bottom support plate 120 (i.e., towards the bottom motor 132). Similarly, the bottom motor 132 may rotate the bottom belt 134 anticlockwise over the bottom idler pulley 136 for moving the bottom UV panel 122 towards the second end 138-2 of the bottom support plate 120 (i.e., away from the bottom motor 132). The bottom UV panel 122 may be moved parallel to the bottom rails 124 (or a length of the bottom support plate 120). Upon being moved, the bottom UV panel 122 may slide along the bottom rails 124 via the guide wheels 14 (such as the second guide wheel 130) engaging with the corresponding slots such as the second slot 126.

Different positions of the bottom UV panel 122 between the opposing ends of the bottom support plate 120 may be determined by the controller 16 via sensors. In one embodiment, as illustrated in FIG. 11, the bottom support plate 120 may include a first bottom position sensor 142-1 and a second bottom position sensor 142-2 (hereinafter collectively referred to as the bottom position sensors 142). Each of the bottom position sensors 142 may be mounted to a support bracket coupled or attached to the bottom support plate 120. In one example, the first bottom position sensor 142-1 may be mounted proximate to the first end 138-1 of the bottom support plate 120 (i.e., near the bottom motor 132). The second bottom position sensor 142-2 may be located proximate to the second end 138-2 of the bottom support plate 120 (i.e., near the bottom idler pulley 136). Each of the bottom position sensors 142 may be similar to the top position sensors 110, discussed above.

Each of the bottom position sensors 142 may operate in communication with the controller 16 for determining different positions of the bottom UV panel 122. For example, based on a contact between the first bottom position sensor 142-1 and the bottom UV panel 122, the first bottom position sensor 142-1 may be configured to send a first bottom signal to the controller 16. The first bottom signal may indicate to the controller 16 that the bottom UV panel 122 may be located proximate to the first end 138-1 of the bottom support plate 120 (or the bottom motor 132). Similarly, based on a contact between the second bottom position sensor 142-2 and the bottom UV panel 122, the second bottom position sensor 142-2 may be configured to send a second bottom signal to the controller 16. The second bottom signal may indicate to the controller 16 that the bottom UV panel 122 may be located proximate to the second end 138-2 of the bottom support plate 120 (or the bottom idler pulley 136). When no signal may be received from any of the bottom position sensors 142, the controller 16 may determine the bottom UV panel 122 being located in-between the opposing ends of the bottom support plate 120.

The bottom UV panel 122 may be configured to project the germicide such as UV light. In one embodiment, as illustrated in FIG. 10, the bottom UV panel 122 may include a bottom mounting plate 144 and a bottom germicidal source 116-2 mounted thereto. In some examples, the bottom UV panel 122 may also include a fan (not shown) mounted thereto for cooling the bottom germicidal source 116-2 and other surrounding components. The bottom mounting plate 144 may have one side including the bottom germicidal source 116-2 and the other side movably mounted to the bottom rails 124 via the shouldered screws such as the second shouldered screw 128, as discussed above. The bottom mounting plate 144 may assist in easy wiring, maintenance, and replacement of the bottom germicidal source 116-2 therefrom. The bottom UV panel 122 including the bottom germicidal source 116-2 may be oriented towards the tray 46 inside the disinfection chamber 40. As illustrated, the bottom germicidal source 116-2 includes strips of one or more UV light emitting diodes (LEDs) configured to emit pulses of UV light; however, some examples may include UV lamps or bulbs configured for pulsed or continuous emission of the UV light. In some other examples, the bottom germicidal source 116-2 may additionally include other types of radiation sources and/or non-radiation sources providing the complementing agents, such as those discussed above. In one embodiment, the bottom UV panel 122 (or the bottom germicidal source 116-2) may be configured to move in a direction, e.g., along the horizontal axis, z-axis, orthogonal to the top UV panel 112 (or the lift assembly 80). For example, the bottom UV panel 122 (and hence, the bottom germicidal source 116-2) may be moved parallel to the bottom rails 124 (or a length of the bottom support plate 120). In some examples, the bottom UV panel 122 (and hence, the bottom germicidal source 116-2) may be configured to move while the top UV panel 112 (or the top germicidal source 116-1) may be moving along the x-axis or the y-axis. Some embodiments may include the bottom UV panel 122 (or the bottom germicidal source 116-2) being configured to move parallel to the top UV panel 112 (or top germicidal source 116-1). Other embodiments may include both the bottom UV panel 122 (or the bottom germicidal source 116-2) and the top UV panel 112 (or top germicidal source 116-1) configured to move along the same horizontal axis or the same vertical axis inside the disinfection chamber 40.

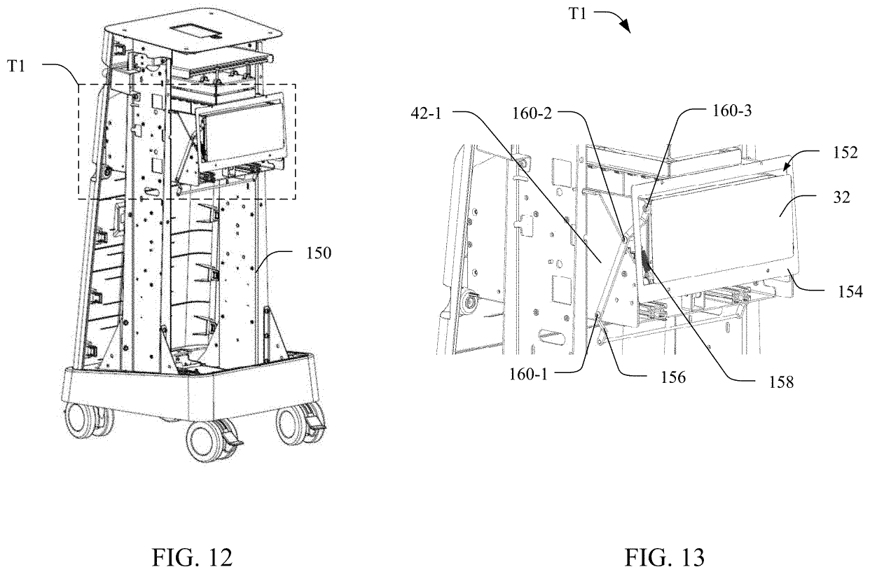

The disinfection chamber 40 may be mounted to the mobile body 12. In one example, as illustrated in FIG. 12, the disinfection chamber 40 may be removably mounted to an uniframe 150 of the mobile body 12 for easy maintenance and replacement of faulty or worn-out components. The uniframe 150 may serve as an integral frame of the mobile body 12 to mount or support various operational components of the disinfection apparatus 10. The uniframe 150 may have a bottom portion attached to the wheels 14 (or the autonomous mobile base including the wheels 14). In some examples, the uniframe 150 may also include one or more vertical trays (not shown) configured to mount or support the control system and other components of the disinfection apparatus 10. Some examples may include vertical or horizontal trays (not shown) configured as storage compartments in the uniframe 150. The mounted disinfection chamber 40 may include the tray 46 aligned with the door 32 and the front opening of the cabinet 20 (or the disinfection apparatus 10).

In one embodiment, the door 32 may be movably coupled to the side plates 42 of the disinfection chamber 40. The door 32 may be positioned within a cut-out 152 of a door plate 154 located parallel to the front side 22 of the cabinet 20 (or the disinfection apparatus 10); however, some examples may include the door 32 being positioned directly in to the front opening of the cabinet 20 (or the disinfection apparatus 10) without the door plate 154. Other examples may include the door plate 154 or the door 32 installed inside or outside the cabinet 20. The door plate 154 may assist in reducing wear and tear of a cabinet portion, including the front opening, and improving the aesthetics of the cabinet 20. Further, the cut-out 152 may have suitable geometry and dimensions for allowing the door 32 and the tray 46 to pass therethrough. The cut-out 152 may be positioned in the moving path of the tray 46 outside the disinfection chamber 40. The tray 46 may be configured to pass through the cut-out 152 to move in and out of the disinfection chamber 40 and the cabinet 20 (or the disinfection apparatus 10).

The door 32 may be configured to transition between an open position and a closed position (hereinafter collectively referred to as door positions). In the closed position (FIG. 13), the door 32 may substantially block or cover the cut-out 152 of the door plate 154 and the front opening of the cabinet 20 (or the disinfection apparatus 10). The door 32 may be positioned in the moving path of the tray 46 outside the disinfection chamber 40. The door 32 may be oriented perpendicular to a horizontal axis of (i) the door plate 154, (ii) the disinfection chamber 40, (iii) the cabinet 20, and (iv) the disinfection apparatus 10. In the open position (FIG. 19), the door 32 may move away from (i) the moving path of the tray 46 outside the disinfection chamber 40, (ii) the cut-out 152 of the door plate 154, and (iii) the front opening of the cabinet 20 (or the disinfection apparatus 10). The door 32 may be oriented at a set angle ranging from 0 degrees to approximately 5 degrees with respect to the horizontal axis; however, some examples may include the door 32 being oriented substantially parallel to the tray 46 or the horizontal axis in the open position. The door 32 may pivot upwards for moving into the open position; however, some examples may include the door 32 pivoting downwards for moving into the open position.

Further, the door 32 may be configured to pivot about the horizontal axis via a biasing mechanism. In one embodiment, the biasing mechanism may be configured to move the door 32 in different door positions. The biasing mechanism may include support arms, such as a support arm 156, and extension springs, such as an extension spring 158, attached to the door 32. Each of the support arms may be located on opposite lateral sides of the door 32. For the sake of brevity, constructional and functional details of only one of the support arms, e.g., the support arm 156, and one of the extension springs, e.g., the extension spring 158, are discussed here. One having ordinary skill in the art would understand that other support arm (not shown) and the other extension spring (not shown) may also have a construction and function similar to those of the support arm 156 and the extension spring 158, respectively.

As illustrated in FIG. 13, in one example, the support arm 156 may include a first pivot point 160-1, a second pivot point 160-2, and a third pivot point 160-3 (hereinafter collectively referred to as pivot points 160). The third pivot point 160-3 may be located between the first pivot point 160-1 and the second pivot point 160-2. The first pivot point 160-1 may be pivotally attached to one of the side plates 42, such as the first side plate 42-1. The second pivot point 160-2 may include a pivoting joint at which the support arm 156 may be configured to bend or move with respect to the remaining pivot points 160. The second pivot point 160-2 may pivotally communicate with one of the extension springs. For example, the extension spring 158 may have a first end and a second end. The first end may be movably connected to the second pivot point 160-2, and the second end may be movably attached to a lower lateral edge of the door 32. The third pivot point 160-3 may be pivotally attached to an upper lateral edge of the door 32. The third pivot point 160-3 may be located on the horizontal axis about which the door 32 may be configured to rotate or pivot.

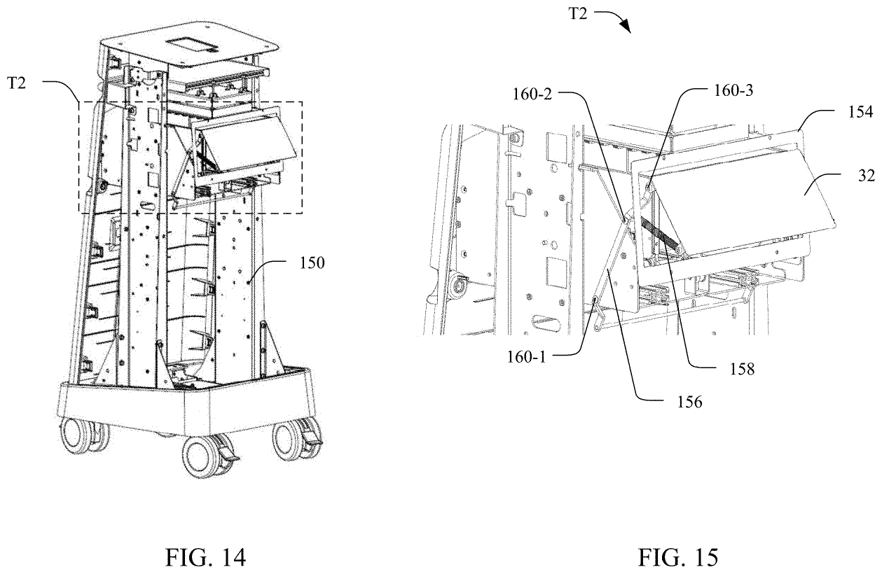

The pivot points 160 in communication with the extension spring 158 may bias the door 32 towards the door positions based on a pivoting direction of the door 32 and the operative positions of the tray 46. For example, when the tray 46 may be set in the non-extended position (FIG. 12), the door 32 may be set in the closed position as shown in FIG. 13. In the non-extended position, the tray 46 may be located within the disinfection chamber 40 inside the cabinet 20 (or the disinfection apparatus 10). The tray 46 may be driven by the tray motor 56 towards the extended position. Upon being driven (FIG. 14), the tray 46 may begin to move out of the disinfection apparatus 10 through the cut-out 152 in the door plate 154 (and/or the front opening of the cabinet 20) while beginning to move door 32 away from (i) the cut-out 152 and/or the front opening.

The tray 46 may push the door 32 outward (FIG. 14) and partially unblock the cutout 152 or the front opening of the disinfection apparatus 10. Due to the push, the door 32 may begin pivoting upwards about the horizontal axis to partially stretch and produce a restoring force in the extension spring 158 connected thereto, as illustrated in FIG. 15. The extension spring 158 may in turn exert a pull force on the second pivot point 160-2 of the support arm 156. The pull force may be balanced by the push force exerted by the door 32 on the third pivot point 160-3, thereby holding the door 32 in-position within the cut-out 152 of the door plate 154 (or the front opening) while allowing the door 32 to rotate or pivot about the horizontal axis. The door 32 movement may assist in clearing the moving path of the tray 46 outside the cabinet 20 (or the disinfection apparatus 10).

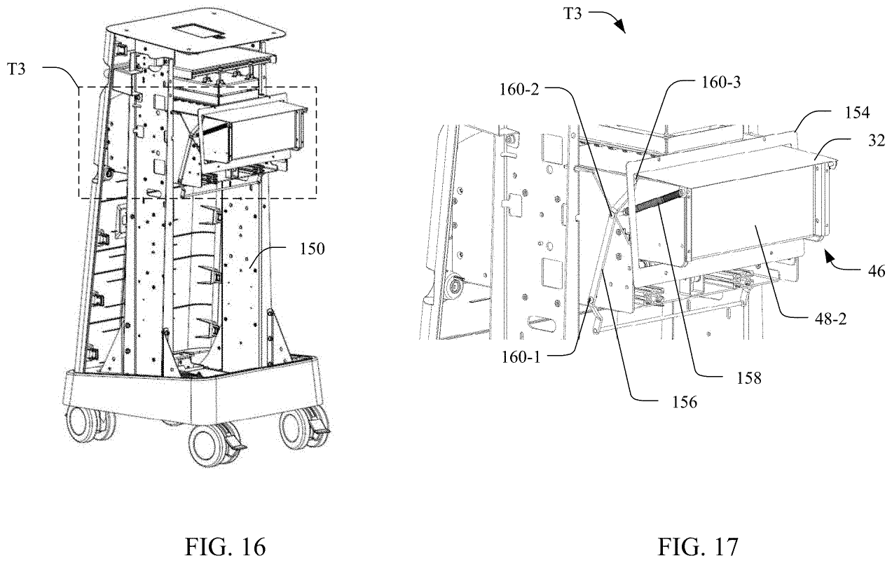

As illustrated in FIG. 16, when the tray 46 may be moved forward or outward to further push the door 32 through the cut-out 152 in the door plate 154 (or the front opening), the door 32 may pivot upwards and towards the open position to unblock the moving path of the tray 46 outside the cabinet 20 (or the disinfection apparatus 10). As illustrated in FIG. 17, the pivoting door 32 may further stretch and increase the restoring force in the extension spring 158 to exert the pull force on the second pivot point 160-2 of the support arm 156. The pull force may be balanced by the push force exerted by the pivoting door 32 on the third pivot point 160-3, thereby holding the door 32 in-position within the cut-out 152 of the door plate 154 while allowing the door 32 to rotate or pivot about the horizontal axis.

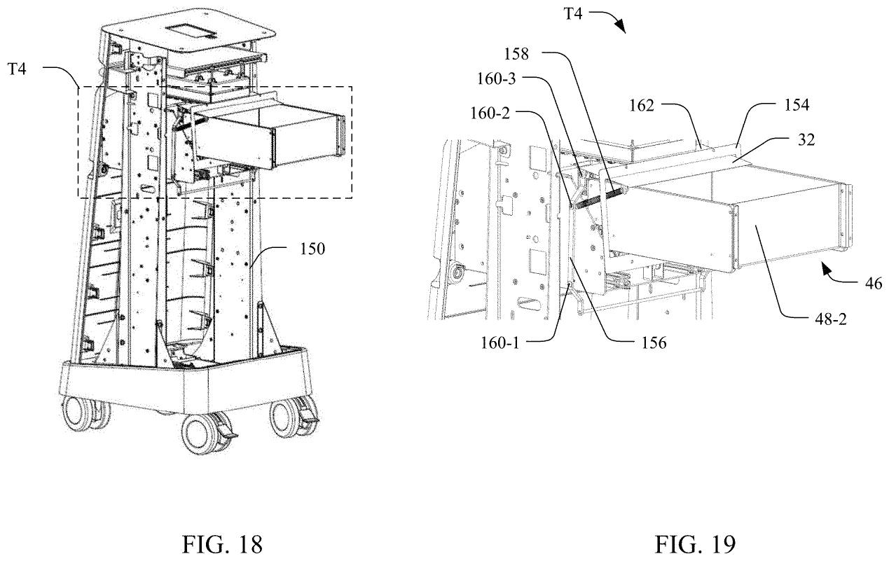

As illustrated in FIG. 18, the tray 46 may be moved fully outward to the extended position through the cut-out 152 in the door plate 154 (or the front opening) while pushing the door 32 to the open position. The door 32 may pivot out of the moving path of the tray 46 outside the cabinet 20 (or the disinfection apparatus 10). As illustrated in FIG. 19, in the open position of the door 32, the push force exerted by the door 32 on the third pivot point 160-3 may exceed the pull force exerted on the second pivot point 160-2 by extension spring. Due to the difference in forces on different pivot points 160 of the support arm 156, the door 32 may move partially inward to have a horizontal portion 162 thereof inserted into the disinfection chamber 40 in the open position. The support arm 156 may pivot about the first pivot point 160-1 to support the inward movement of the horizontal portion 162 of the door 32 in the open position. The inward movement (and the inserted horizontal portion 162) of the door 32 in the open position may assist in avoiding any accidental pinching of fingers of a user from being caught between the door 32 and the cut-out 152 (or the front opening).

Subsequently, the tray 46 may be driven by the tray motor 56 for moving back to the non-extended position inside the disinfection chamber 40. The backward/inward motion of the tray 46 may vacate the cut-out 152 or the front opening to gradually provide a space for the door 32 to pivot downward while gradually releasing or reducing the push force exerted by door 32 on the third pivot point 160-3. The reduction in the push force on the third pivot point 160-3 relative to the pull force exerted on the second pivot point 160-2 may pull the inserted horizontal portion 162 of the door 32 to move out of the disinfection chamber 40. The restoring force in the extension spring 158 may then urge the door 32 to pivot downwards to the closed position after tray 46 may be moved to the non-extended position. In the closed position, the door 32 may block the cut-out 152 of the door plate 154 and the front opening of the cabinet 20 (or the disinfection apparatus 10), thereby preventing access to an interior portion of the cabinet 20 via the front opening of the cabinet 20 (or the disinfection apparatus 10). Hence, the biasing mechanism, including the support arms and the extension springs, implemented as a motor-less mechanical system for the door 32 may assist in regulating access to the interior portion of the cabinet 20 while saving the battery and avoiding any complex hardware and software programming for manipulating the door.

### Operation

In one embodiment, the controller 16 may control various components and functions of the disinfection apparatus 10. During operation, the controller 16 may operate the disinfection apparatus 10 in a normal mode or an automated mode (hereinafter collectively referred to as device modes). In the automated mode, the controller 16 may (i) drive the wheels 14 (or the autonomous mobile base) autonomously or via the remote device for moving or orienting the disinfection apparatus 10 to a target position or orientation, (ii) facilitate remote controlling of the movable germicidal system 18 within the disinfection apparatus 10 via the remote device for activating or inhibiting (or stopping) projection of a germicide such as the UV light, and (iii) selectively control and/or monitor movements of the movable germicidal system 18 and any movable components thereof. In the normal mode, the controller 16 may enable a user to manually move or steer the wheels 14 (or the autonomous mobile base) to a desired spatial location such as a room. In another example, the controller 16 may additionally operate the disinfection apparatus 10 in one of a time mode and an object mode (hereinafter collectively referred to as disinfection modes). In the time mode, the controller 16 may activate the movable germicidal system 18 to move and project the germicide such as the UV light for any predefined or dynamically defined duration (e.g., ranging from approximately 10 seconds to approximately 180 seconds) defining a first disinfection cycle. In the object mode, the controller 16 may activate the movable germicidal system 18 to move and project the germicide such as the UV light for a predefined duration based on a surface type or a property of a received article requiring disinfection (e.g., approximately 20 seconds or less for a non-permeable article such as an electronic device and approximately 30 seconds or less for a permeable article such as a facial mask), thereby defining a second disinfection cycle.

Each of the device modes and the disinfection modes (collectively referred to as the operational modes) may be implemented to operate independently, or in tandem with each other, in any suitable combinations or order. However, some examples may include a particular operational mode being operable mutually exclusive to one or more of the remaining operational modes. For instance, the controller 16 may implement the automated mode based on the disinfection modes and the normal mode being deactivated. In some instances, the controller 16 may automatically deactivate the automated mode based on any of the disinfection modes and the normal mode being selected. These operational modes may be selected by a user using any of the suitable input devices known in the art. For example, the user may login on an input device such as the display unit 34 and an interactive display screen of a remote computing device operating in communication with the controller 16 of the disinfection apparatus 10 to select or deselect any of these operational modes.

When the disinfection apparatus 10 may be powered-on, the tray 46 may be set in the non-extended position, the door 32 may be set in the closed position, and the top germicidal assembly 70 including the top UV panel 112 may be set in the retracted state. In the non-extended position, the tray 46 may be substantially positioned within the disinfection chamber 40, e.g., inside the cabinet 20. The door 32 may be set in the closed position to block the moving path of the tray 46 and the front opening of the cabinet 20 (or the disinfection apparatus 10). In the closed position, the tray 46 as well as the door 32 may be located inside the cabinet 20; however, some examples may include the door 32 located outside the cabinet 20 or within a vertical plane (or a slanted plane) comprising the cabinet 20. Each of the top germicidal assembly 70 and the bottom germicidal assembly 72 may be inactive upon powering on the disinfection apparatus 10. Moreover, the top germicidal assembly 70 may include the top UV panel 112 in the retracted state and the bottom UV panel 122 positioned vertically thereunder defining a default position when the disinfection apparatus 10 may be powered on.

The controller 16 may control movements of the tray 46 in response to an input signal from one or more sensors, the display unit 34, and/or the remote device. Based on the input signal, the tray 46 may be configured to transition between the non-extended position and the extended position. The controller 16 may receive the input signal based on predefined or dynamically defined conditions. For example, the controller 16 may be configured to drive the tray base 50d on signals received from the sensors such as the top sensor 64 and the front sensor 66. Both the top sensor 64 and the front sensor 66 may be configured to provide signals indicating a value ‘1’ to the controller 16 upon detecting any obstruction or motion within predefined or dynamically defined distances (e.g., up to at least approximately 15 cm) therefrom and otherwise indicating a value ‘0’ to the controller 16. The sensor signals may be received as a signal set (X, Y) where X, or a first value, may refer to a value indicated by a first signal from the top sensor 64 and Y, or a second value, may refer to a value indicated by a second signal from the front sensor 66. For instance, in the absence of any such obstruction or motion, the controller 16 may receive the signal set (0,0) from the top sensor 64 and the front sensor 66 respectively. In one embodiment, the controller 16 may be configured to activate the tray motor 56 for moving the tray base 50d on the first signal indicating a value ‘1’ and the second signal indicating a value ‘0’ in the signal set. The value ‘1’ from the top sensor 64 may be provided, e.g., based on a user waving his/her hand across the top sensor 64, thereby supporting a touchless operation of the disinfection apparatus 10. However, other examples may include the top sensor 64 providing the first signal upon sensing a unique signature (e.g., fingerprint, retina, a symbol such as tattoo and barcode, a hand gesture, a finger gesture, magnetic strip or data card, etc.) provided by a user. The value ‘0’ from the front sensor 66 may indicate a clear moving path being available up to a predefined minimum distance being available outside the cabinet 20 (or the disinfection apparatus 10) for extending the tray 46 to the extended position without any obstructions. In one example, the minimum distance may be at least one-fourth of a length of the tray 46; however, other examples may include the minimum distance ranging from approximately 4 inches to approximately 25 inches. Accordingly, the controller 16 may begin driving the tray 46, via the tray motor 56, towards the extended position upon receiving the signal set (1, 0). In some embodiments, the controller 16 may also begin driving the tray 46 towards the extended position based on a received signal set being (0, 0) when the disinfection apparatus 10 may be powered on.

Based on the received signal set, the controller 16 may the drive the tray motor 56 for moving the tray 46 towards the extended position. The moving tray 46 may push the door 32 outward to unblock the cut-out 152 or the front opening of the disinfection apparatus 10. Due to the push, the door 32 may pivot upwards to move away from the moving path of the tray 46, as discussed above. During movements of the tray 46, the rear sensor may monitor positions of the tray 46 relative to the disinfection chamber 40. In some embodiments, the controller 16 may change the previously received value of the first signal from ‘1’ to ‘0’ after the controller 16 may have triggered the tray motor 56 for driving the tray 46 to the extended position. Such change in the received signal value from the top sensor 64 may assist in reusing the top sensor 64 for transitioning the tray 46 from the extended position to the non-extended position based on a user input, e.g., the top sensor 64 sensing a hand of the user, thereby further automating a touchless operation of the disinfection apparatus 10.

In some embodiments, the controller 16 may cease to drive the tray motor 56 to stop any motion of the tray 46 if the front sensor 66 sends a signal indicating a value ‘1’ while the tray 46 may be transitioning to the extended position. The value ‘1’ from the front sensor 66 may indicate that the moving path of the tray 46 outside the cabinet 20 (or the disinfection apparatus 10) may be unavailable or not clear up to the predefined minimum distance for the tray 46 to move to the extended position without any obstructions. At this point, the controller 16 may automatically trigger a backward/inward motion of the tray 46 to transition back to the non-extended position within the disinfection chamber 40 based on the front sensor 66 detecting such obstruction or motion. Additionally, or alternatively, the controller 16 may provide an indication (e.g., pulsating light, beep, vibration, etc.) for a user to clear the moving path and provide the predefined minimum distance for allowing the tray 46 to move outside the cabinet 20 (or the disinfection apparatus 10). In further embodiments, the controller 16 may be configured to keep the tray 46 in the extended position substantially outside the cabinet 20 (or the disinfection apparatus 10) until a signal indicating the value ‘1’ may be received from at least one of the top sensor 64 and the front sensor 66. Other embodiments may include the controller 16 being configured to keep the tray 46 in the extended position for a predefined or dynamically defined duration (e.g., ranging from approximately 5 seconds to approximately 20 seconds) at the end of which the controller 16 may automatically trigger the tray motor 56 for retracting the tray 46 to the non-extended position within the disinfection chamber 40.

As illustrated in FIG. 20, the tray 46 may be positioned in the extended position for receiving an article 170 requiring disinfection. Thereafter, the controller 16 may drive the tray 46, via the tray motor 56, to move backward or inward for being positioned within the disinfection chamber 40. The backward/inward motion of the tray 46 may cause the door 32 to pivot downwards to the closed position, thereby blocking the front opening and any access therethrough to the disinfection chamber 40 and/or any interior portion of the cabinet 20, as discussed above. The rear sensor may sense the tray 46 positioned in the non-extended position, as illustrated in FIG. 21, and may send a third signal, e.g., indicating a value ‘1,’ to the controller 16. Based on the third signal, the controller 16 may activate a predefined disinfection cycle, such as those discussed above, for disinfecting the received article 170. In some examples, a user may select one of the first disinfection cycle and the second disinfection cycle via the display unit 34 or the remote device.

During the disinfection cycle, the controller 16 may activate the top germicidal source 116-1 and the bottom germicidal source 116-2 (hereinafter collectively referred to as germicidal sources 116) to project the germicide such as the UV light to disinfect the article 170 placed in the tray 46. In the illustrated example (FIG. 21), the germicidal sources 116 may be configured to project the UV light for the predefined disinfection cycle with the top UV panel 112 in the retracted state and the bottom UV panel 122 either stationarily located vertically thereunder or moving parallel thereto, as discussed above. The controller 16 may drive the germicidal sources 116 to irradiate timed pulses of the germicide, such as UV light, with each pulse having predefined characteristics such as energy, power, wavelength, and/or frequency. For example, the controller 16 may drive each of the germicidal sources 116, simultaneously or alternately, at a predefined or dynamically defined pulse frequency (e.g., approximately 50 Hz) to emit an intended amount of energy per pulse. In another example, the controller 16 may drive the germicidal sources 116 at a combined pulse frequency of at least 10 Hz to emit a predefined amount of energy. In yet another example, the controller 16 may drive the top germicidal source 116-1 and the bottom germicidal source 116-2 to (i) emit the same or different types of germicides at the same or different frequencies. For instance, at least one of the germicidal sources 116 may include multiple UV LEDs with (i) each UV LED having a total optical power output ranging from approximately 50 mW to approximately 80 mW, (ii) a combined energy output ranging from approximately 10 J/cm² to approximately 20 J/cm², an energy per pulse ranging from 20 to 150 Joules, and a pulse frequency ranging from 2 Hz to 60 Hz. In some other examples, the controller 16 may trigger the germicidal sources 116 alternately to collectively emit the germicide, such as the UV light, on to the article 170, or the tray 46, in a pulsed manner. The controller 16 may toggle or switch from driving one of the germicidal sources 116 to the other at a predefined or dynamically defined toggling rate to emit the germicide such as the UV light within the disinfection cycle. In some instances, the toggling rate may be set based on the pulse frequency and/or the energy per pulse associated with at least one of the germicidal sources 116.

In one embodiment, the controller 16 may detect the article 170 within the disinfection chamber 40 using the top position sensor mounted to the top germicidal assembly 70. For example, the controller 16 may manipulate the linear actuator 86 of the lift assembly 80 in the top germicidal assembly 70. The manipulated linear actuator 86 may extend the articulated arm 84 in the lift assembly 80 for repositioning the top UV panel 112. The articulated arm 84 may extend downwardly along the vertical axis (e.g., y-axis) for repositioning the top UV panel 112 into the extended state, thereby moving the top UV panel 112 closer to the article 170 at a set distance therebetween for improving the rate of article disinfection. For instance, the extending articulated arm 84 may move or lower the top UV panel 112 orthogonal to (i) a direction of motion of the tray 46, (ii) the tray 46 (or the tray base 50) carrying or supporting the article 170, and (iii) the bottom UV panel 122 or a direction of motion thereof. However, some examples may include the articulated arm 84 implemented as the pivotable telescopic arm pivoting at a predefined pivot angle while moving or lowering the top UV panel 112 towards the tray base 50 or the received article 170, for detecting the received article 170. The pivot angle may range from 0 degrees to approximately 90 degrees about an arm joint in the articulated arm 84. In some examples, the arm joint may have at least two degrees of freedom (dof) or more depending on a configuration of the articulated arm 84.

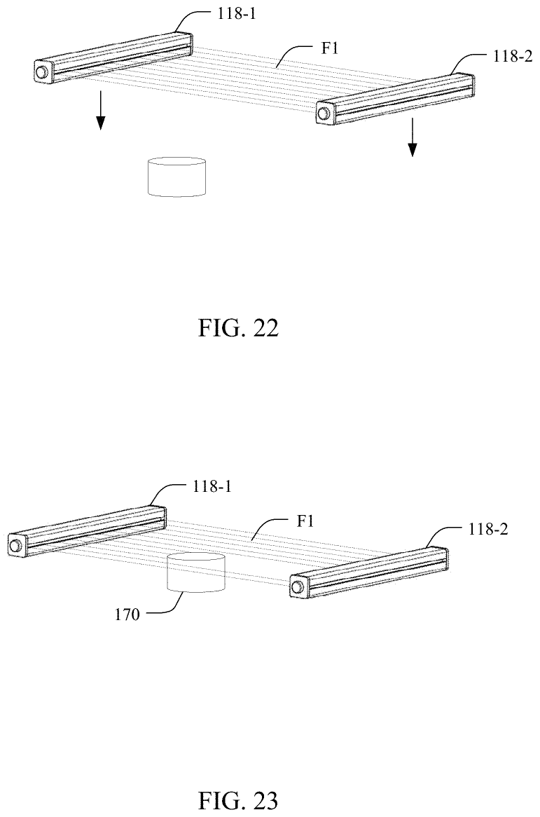

As illustrated in FIG. 22, the controller 16 may extend the articulated arm 84, via the linear actuator 86, until the received article 170 intercepts the continuous optical field F1 between the optical sensors 118. Based on the interception (FIG. 23), in one example, the optical receiver 118-2 may send the detection signal to the controller 16; however, other examples may include the optical transmitter 118-1 sending the detection signal to the controller 16 based on such interception or interference. The detection signal (and the interference) may indicate a presence (or detection) of the article 170 to the controller 16. Based on the article 170 being detected, the controller 16 may stop the extension of the articulated arm 84 and hold the top UV panel 112 close to the article 170, thereby maintaining a set distance between a surface of the article 170 and the top UV panel 112 (or the top germicidal source 116-1). In one example, the set distance may be approximately 20 mm; however, other examples may include the set distance ranging from approximately 20 mm to approximately 150 mm.

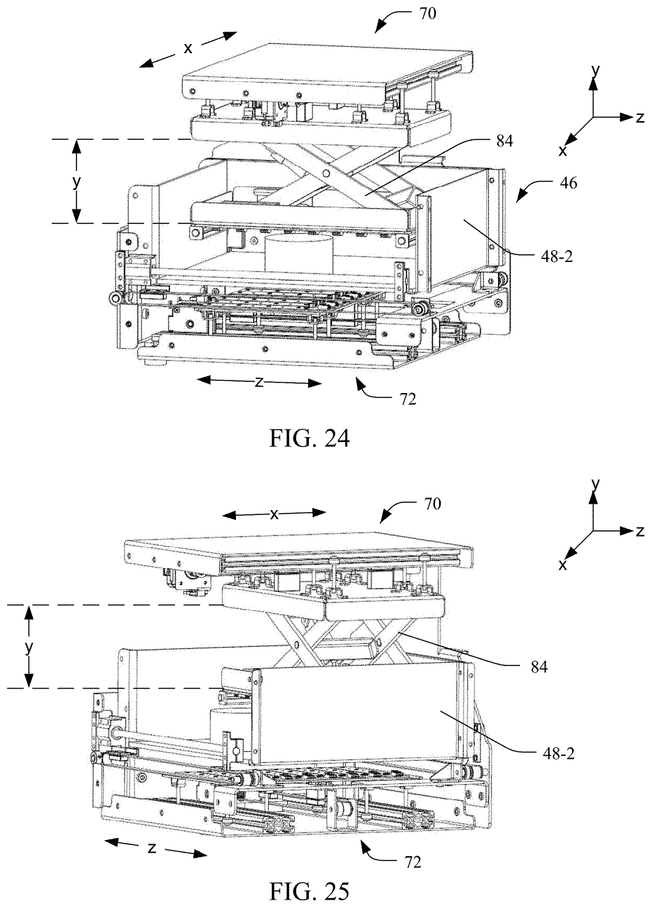

While the top UV panel 112 is being moved closer to the article 170, the controller 16 may activate the bottom motor 132 to move the bottom UV panel 122 (and the bottom germicidal source 116-2) back and forth along the bottom rails 124 via the bottom belt 134, as illustrated in FIG. 24. In one example, the bottom UV panel 122 (and the bottom germicidal source 116-2) may be moved in the horizontal axis (e.g., z axis) orthogonal to the vertical axis (e.g., y axis) along which the articulated arm 84 may extend (or retract) the top UV panel 112 (and the top germicidal source 116-1) relative to the tray base 50 and/or the article 170, thereby improving the surface coverage of the article 170 by the germicide such as the UV light while eliminating shadowing of the article surface.

Further, the controller 16 may also activate the top motor 100 to move the lift assembly 80 carrying the top UV panel 112 (and the top germicidal source 116-1) back and forth along the top rails 92 via the top belt 102, as illustrated in FIG. 25. In one example, the lift assembly 80 carrying the top UV panel 112 (and the top germicidal source 116-1) may be moved in a horizontal axis (e.g., x axis) orthogonal to the horizontal axis (e.g., z axis) of motion of the bottom UV panel 122 as well as the vertical axis (e.g., y axis) of motion/extension of the articulated arm 84 of the lift assembly 80. Hence, the motion of the top UV panel 112 carrying the top germicidal source 116-1 along the two axes (e.g., y-axis and x-axis) while moving the bottom UV panel 122 along the orthogonal axis (e.g., z-axis) may assist in projecting the germicide such as the UV light on all sides or surfaces of the article 170 simultaneously to improve the rate of article disinfection and reduce the time required for complete or adequate article disinfection.

After completion of the disinfection cycle, the controller 16 may (i) retract the articulated arm 84 via the linear actuator 86 for repositioning the top UV panel 112 back to the retracted state, (ii) stop the back-and-forth motions of the top UV panel 112 and the bottom UV panel 122, and (iii) deactivate the top germicidal source 116-1 as well as the bottom germicidal source 116-2. Thereafter, the controller 16 may drive the tray 46, via the tray motor 56, to transition from the non-extended position to the extended position outside the disinfection chamber 40 and the disinfection apparatus 10 to enable retrieval of the disinfected article 170 from the tray 46. The rear sensor may send the third signal, e.g., indicating a value ‘0’ to the controller 16, thereby indicating the tray 46 being positioned in the extended position. Other embodiments may include the controller 16 programmed to keep the tray 46 in the non-extended position at the end of the disinfection cycle until the signal set (1, 0) may be received from the top sensor 64 and the front sensor 66 respectively. The top sensor 64 may send such first signal indicating the value ‘1’ based on a user input, e.g., a user hand being detected by the top sensor 64, thereby further automating the touchless operation of the disinfection apparatus 10 to access the tray 46 and collect the disinfected article 170.

Various kinds, sizes, shapes, and materials of various components including those not necessarily depicted in the attached drawings may also be envisaged by invention(s) covered in the present application. Notably, the figures and examples are not meant to limit the scope of the present application to a single embodiment, but other embodiments are possible by way of interchange of some or all of the described or illustrated elements.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiments, methods, and examples, but by all embodiments and methods within the scope and spirit of the invention(s).

I/We claim:

1. An apparatus for disinfecting an article, the apparatus comprising:
a disinfection chamber inside a cabinet;
a tray movably attached to the disinfection chamber, wherein the tray is moveable between a first position outside the cabinet for placement and retrieval of an article in the tray and a second position within the disinfection chamber for disinfection of the article; and
a first germicidal assembly movably mounted to the disinfection chamber, the first germicidal assembly including an articulated arm and a first UV panel mounted thereto, wherein the articulated arm is configured to extend the first UV panel in a first direction towards the article inside the cabinet for disinfecting the article.

2. The apparatus of claim 1, wherein at least one of the articulated arm and the first UV panel is configured to move in a second direction orthogonal to the first direction.

3. The apparatus of claim 2, further comprises a second germicidal assembly movably mounted to the disinfection chamber, wherein the second germicidal assembly includes a second UV panel configured to move in a third direction orthogonal to at least one of the first direction and the second direction.

4. The apparatus of claim 3, wherein the second germicidal assembly is mounted under the tray.

5. The apparatus of claim 3, wherein the first UV panel and the second UV panel are configured to project UV light towards the tray, wherein the first UV panel and the second UV panel are triggered alternately by a controller to collectively emit UV light in a pulsed manner on to the article.

6. The apparatus of claim 1, wherein the first UV panel is configured to project pulsed UV light.

7. The apparatus of claim 1, wherein the disinfection chamber is distinct from the cabinet.

8. The apparatus of claim 1, wherein the first germicidal assembly is mounted above the tray.

9. The apparatus of claim 1, wherein the tray includes a base for supporting the article, wherein the base is optically permeable to UV light.

10. The apparatus of claim 1, wherein the articulated arm is configured to extend the first UV panel up to a distance of at least 150 mm from the article.

11. A method of disinfecting an article, the method comprising:
providing a disinfection chamber inside a cabinet, wherein the disinfection chamber includes a tray moveable between a first position outside the cabinet for placement and retrieval of an article in the tray and a second position within the disinfection chamber for disinfection of the article; and
manipulating, via a controller, a first germicidal assembly movably mounted to the disinfection chamber, the first germicidal assembly including an articulated arm and a first UV panel mounted thereto, wherein the articulated arm is manipulated to extend the first UV panel in a first direction towards the article inside the cabinet for disinfecting the article.

12. The method of claim 11, wherein the step of manipulating further comprises moving at least one of articulated arm and the first UV panel in a second direction orthogonal to the first direction.

13. The method of claim 12, further comprising manipulating a second germicidal assembly including a second UV panel movably mounted to the disinfection chamber, wherein the second UV panel is manipulated to move in a third direction orthogonal to at least one of the first direction and the second direction.

14. The method of claim 13, wherein the second UV panel is manipulated to move under the tray.

15. The method of claim 13, further comprising operating, using the controller, the first UV panel and the second UV panel to project UV light alternately on to the article.

16. The method of claim 11, wherein the disinfection chamber is distinct from the cabinet.

17. The method of claim 11, wherein the first germicidal assembly is mounted above the tray.

18. The method of claim 11, wherein the tray includes a base for supporting the article, wherein the base is optically permeable to the UV light.

19. The method of claim 11, wherein the articulated arm extends the first UV panel up to a distance of at least 150 mm from the article.

20. The method of claim 11, wherein the articulated arm includes at least one of an articulating scissor arm, a telescopic arm, and a pivotable telescopic arm.
